(19) United States
(12) Patent Application Publication
(10) Pub. No.: US 2002/0169288 A1
(43) Pub. Date: Nov. 14, 2002

(54) Collagen-binding adhesin from staphylococcus epidermidis and method of use

(76) Inventors: Magnus Hook, Houston, TX (US); Maria Bowden, College Station, TX (US)

Correspondence Address: LARSON & TAYLOR, PLC, 1199 NORTH FAIRFAX STREET, SUITE 900, ALEXANDRIA, VA, 22314, US

(21) Appl. No.: 10/098,174

(22) Filed: Mar. 15, 2002

Related U.S. Application Data
(60) Provisional application No. 60/275,718, filed on Mar. 15, 2001.

(51) Int. Cl. C07K 1/00; C07K 14/00; C07K 17/00

(52) U.S. Cl. 530/350

(57) ABSTRACT

An Isolated lipase, designated GehD, from S. epidermidis has now been found to bind to collagen, and thus this protein may be used in methods of preventing or treating staphylococcal infections. The invention contemplates the use of compositions including GehD, vaccines containing GehD, and antibodies that can recognize GehD, and these are advantageously used with human or animal patients which may be susceptible to staphylococcal disease. In addition, medical instruments or biological implants can be treated using the collagen-binding protein of the invention in order to reduce or eliminate the possibility of their becoming infected or further spreading a staphylococcal infection.

### CROSS-REFERENCE TO RELATED APPLICATIONS

This application claims the benefit of U.S. Provisional Application Ser. No. 60/275,718 filed Mar. 15, 2001.

### FIELD OF THE INVENTION

The present invention relates in general to a collagen-binding adhesin from Staphylococcus epidermidis, and in particular to the isolation and use of the GehD lipase from S. epidermidis which demonstrates collagen- and laminin-binding abilities and which can thus be utilized in methods of prevention or treatment of diseases causes by staphylococcal pathogens such as S. epidermidis.

### BACKGROUND OF THE INVENTION

Staphylococcus epidermidis is now recognized as an important nosocomial pathogen. In the past twenty years it has emerged as a frequent cause of infections associated with indwelling devices such as catheters, artificial heart valves and orthopedic implants (2). In certain populations, such as low birth weight infants and immuno-compromised patients, S. epidermidis can be a prominent source of morbidity and mortality (de Silva et al 2001).

The molecular mechanisms of pathogenesis of S. epidermidis disease are not well understood, but, as with most infections, bacterial adherence to host surfaces is recognized as the first crucial step in the infection process and a prerequisite for colonization. A two-step process of S. epidermidis adherence is often described, in which the first step is bacterial attachment to the biomaterial, and the second step includes microbial proliferation, intercellular adhesion and biofilm formation. Almost all S. epidermidis strains are able to attach to native abiotic surfaces (9) (17) (21) (14). However, to decipher the initial steps in S. epidermidis foreign body infections, it is critical to understand that any foreign material implanted into the human body is quickly coated with various plasma proteins, such as Fg, Fn, and vitronectin (8) (23).

The adherence properties of S. epidermidis suggest that this organism expresses MSCRAMM®s (Microbial Surface Components Recognizing Adhesive Matrix Molecules). In fact, it has now been known to obtain genes encoding a fibrinogen-binding MSCRAMM via cloning and sequencing from S. epidermidis (15). This 119 kDa fibrinogen binding MSCRAMM has a structural organization similar to the clumping factor (ClfA) from S. aureus. In addition, the autolysin AtlE, necessary for S. epidermidis attachment to polystyrene, was shown to specifically bind to biotin-labeled vitronectin (7). Still further, polypeptides and polynucleotides have been obtained from coagulase-negative staphylococci, and some of these polypeptides such as the SdrG protein have been shown to bind to fibrinogen, such as disclosed in pending U.S. application Ser. No. 09/386,962, filed Aug. 31, 1999, incorporated herein by reference. Overall, these data indicate that S. epidermidis, similarly to S. aureus, may have specific MSCRAMM®s that mediate cell-attachment to host protein-conditioned surfaces.

However, because of the uncertainty involved in accurately determining the nature of the surface binding proteins of S. epidermidis and other adhesins, it is highly desirable to isolate and identify proteins which can be shown to bind to surface proteins such as collagen. Moreover, since antibodies generated from these surface proteins can vary greatly and have a range of effectiveness in inhibiting binding of bacteria to host cells and biological or medical materials and implants, it is important to identify and isolate binding proteins which can generate antibodies that will be effective in blocking such binding and which be useful in methods of treating or preventing diseases caused by staphylococcal bacteria.

### SUMMARY OF THE INVENTION

Accordingly, it is an object of the present invention to identify and isolate binding proteins which can generate antibodies that will be effective in blocking such binding and which be useful in methods of treating or preventing diseases caused by staphylococcal bacteria.

It is an also object of the present invention to isolate and purify GehD lipase, a collagen adhesin from S. epidermidis, which can be used to generate antibodies and compositions which can be effective in blocking staphylococcal adhesion to collagen.

It is further an object of the present invention to provide and utilize compositions based on GehD to treat or prevent infection from staphylococcal bacteria.

It is still further an object of the present invention to provide a vaccine based on GehD which can be used in treating or preventing infection by staphylococcal bacteria such as S. epidermidis.

It is still further an object of the present invention to generate antisera and antibodies to the collagen binding GehD adhesin from S. epidermidis which also be used in the treatment or prevention of bacterial infection, and which can be used to prevent against bacterial infection in biological implants.

It is also an object of the present invention to provide improved materials and methods for detecting and differentiating collagen-binding proteins in staphylococcal organisms in clinical and laboratory settings.

These and other objects are provided by virtue of the present invention which comprises identifying, isolating and/or purifying the GehD lipase adhesin from staphylococcal bacteria such as S. epidermidis which has now been shown to possess collagen and laminin-binding properties and which can thus be useful in methods of treating or protecting against staphylococcal disease. In addition, suitable compositions can be made utilizing the GehD lipase including vaccines which contain an immunogenic amount of GehD, and antibodies that are raised against and thus can bind to GehD which have been shown to be effective in blocking staphylococcal adherence to collagen, and which can thus be used in effective amounts so as to treat or prevent a staphylococcal infection.

These embodiments and other alternatives and modifications within the spirit and scope of the disclosed invention will become readily apparent to those skilled in the art from reading the present specification and/or the references cited herein.

### BRIEF DESCRIPTION OF THE DRAWING FIGURES

FIG. 1 is a graphic representation showing that S. epidermidis can bind to immobilized extracellular matrix proteins. For the graph shown in the figure, Log-phase bacterial cultures were washed and incubated in microtiter wells coated with 10 μg of bovine serum albumin, collagen I, fibronectin and fibrinogen. Attached S. epidermidis cells were detected using crystal violet.

FIG. 2 is a graphic comparison of the reactivity of mAbs generated against CNA to S. epidermidis strains. To obtain these results, microtiter wells were coated with 2 μg of human fibronectin (HFn), washed, blocked with a solution of 2% BSA. The wells were incubated for 2 hours at 22° C. with 100 μl of 2% BSA containing 1×10⁸ cells of S. epidermidis or S. aureus Cowan 1 Δspa::Tc^{R}. Unbound cells were removed by washing, and bound cells were incubated for 2 hours at 22° C. with 2 μg of the indicated mAbs. After extensive washing with PBST, bound antibody was detected peroxidase-conjugated rabbit anti-mouse IgG diluted 1:500. The conjugated enzyme was reacted with o-phenylenediamine dihydrochloride, and the absorbance at 492 nm was measured with a microplate reader.

FIG. 3 again represents staining procedure results showing that mature GehD binds to collagen. Recombinant MGehD was overexpressed and purified using standard techniques. MGehD was separated by SDS-PAGE. Lanes 1 and 2 were stained with Coomassie brilliant blue, lanes 3, 4 and 5 were transferred to a nitrocellulose membrane and probed with digoxigenin-labeled collagen or monoclonal antibodies.

FIG. 4 is a graphic representation showing the far-UV CD spectra of recombinant, mature GehD. The predicted GehD secondary structure composition is reported in the text. Mean residue weight ellipticity is reported in degrees cm²/dmol.

FIG. 5 is a graphic representation of the binding of recombinant, mature GehD to immobilized collagens. Microtiter wells were coated with 10 μg of collagen I (●), II (▪), IV (▴) or BSA (∘). Increasing concentrations of biotinylated, recombinant GehD were incubated in the wells for 1 h at room temperature. Bound protein was detected with AP-conjugated streptavidin, followed by development with p-nitrophenylphosphate substrate. Values represent the means of triplicate wells. This experiment was repeated three times with similar results.

FIG. 6 is a graphic representation showing the Inhibition of GehD binding to immobilized collagen. Recombinant, biotinylated MGehD was pre-incubated with anti-GehD (▪) or pre-immune (●) antibodies before it was incubated in microtiter wells coated with 10 μg collagen I. Biotinylated, bound protein was detected with avidin conjugated to alkaline phosphatase.

FIG. 7. is a graphic representation showing the inhibition of S. epidermidis strains binding to type I collagen by GehD mature domain. Microtiter wells were coated with 10 μg of type I collagen, washed and preincubated for 1 h at room temperature with increasing concentrations of recombinant, mature GehD. Log-phase S. epidermidis 9 (●) and S. epidermidis 9 gehD::ermC (▪) cultures were washed and added to the coated wells. Attached cells were detected staining the cells with crystal violet and measuring their absorbance at 590 nm. Values represent the means and standard deviations of triplicate wells. This experiment was repeated several times with similar results.

FIG. 8 is a graphic representation showing the inhibition of S. epidermidis strains binding to type I collagen by anti-GehD mature domain. Log-phase S. epidermidis 9 (●) and S. epidermidis 9 gehD::ermC (▪) cultures were washed and preincubated with anti-mature-GehD IgG before addition to wells coated with type I collagen. Attached cells were detected staining the cells with crystal violet and measuring their absorbance at 590 nm. Pre-immune IgGs did not inhibit the attachment (data not shown). The experiment was performed in triplicate, the mean values are shown.

### DETAILED DESCRIPTION OF THE PREFERRED EMBODIMENTS

In accordance with the present invention, there is provided an isolated and/or purified GehD lipase adhesin from S. epidermidis which has now unexpectedly been shown to bind to collagen. In addition, in accordance with the invention, pharmaceutical compositions can be prepared which comprise GehD lipase and a pharmaceutically acceptable vehicle, carrier or excipient, and vaccines can be prepared which comprise an immunogenic amount of GehD lipase is a suitable vehicle, carrier or excipient. Further, as set forth in more detail below, methods of utilizing the GehD lipase protein to block the adherence of staph bacteria to collagen are also provided, in additions to methods of treating or preventing a staphylococcal infection by administration of an effective amount of a GehD composition to a human or animal patient in need of such treatment. Finally, antibodies to GehD can be generated which can be useful in methods of treating or preventing staphylococcal infection, and such antibodies can be used, such as in kits with suitable means to identify binding, to determine the presence of the GehD protein and obtain information concerning the nature of an infection.

The GehD lipase from S. epidermidis is described and sequenced in Longshaw et al., Microbiology 146:1419-1427 (2000), incorporated herein by reference. As shown therein, GehD appears to be translated as a 650-700amino acid precursor which is processed post-translationally to an extracellular mature lipase (or MgehD) of 360 amino acids with a size of approximately 45 kDa. However, at the time that this enzyme was first isolated, there was no reason to have expected that it would have collagen binding properties such as has been discovered and utilized in accordance with the present invention.

In addition, a recent sequence analysis of the GehD protein was done, and this sequence is shown as SEQ ID NO: 1. However, as would be recognized by one of ordinary skill in this art, modification and changes may be made in the structure of the peptides of the present invention and DNA segments which encode them and still obtain a functional molecule that encodes a protein or peptide with desirable characteristics. The amino acid changes may be achieved by changing the codons of the DNA sequence. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions, or corresponding DNA sequences which encode said peptides without appreciable loss of their biological utility or activity.

In addition, amino acid substitutions are also possible without affecting the collagen binding ability of the isolated proteins of the invention, provided that the substitutions provide amino acids having sufficiently similar properties to the ones in the original sequences.

Accordingly, acceptable amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine. The isolated proteins of the present invention can be prepared in a number of suitable ways known in the art including typical chemical synthesis processes to prepare a sequence of polypeptides.

The identification and isolation of GehD in accordance with the present invention may proceed via conventional techniques well within the scope of one of ordinary skill in this art. In one such suitable procedure, the GehD lipase may be produced recombinantly by obtaining a nucleic acid fragment encoding the GehD lipase or its mature domain, such as through the construction of an expression library from a suitable vector such as an S. epidermidis 9491 λZAP Express (Stratgene) expression library following by amplification of the nucleic acid fragment via PCR or other appropriate process. The proteins are thus produced recombinantly from the obtain nucleic acids and can be isolated using appropriate agents such as biotin. It is also possible to isolate and/or purify natural GehD from S. epidermidis if so desired.

The proteins of the invention can thus be prepared using the well known techniques of solid phase, liquid phase, or peptide condensation techniques, or any combination thereof, and can include natural and unnatural amino acids. Amino acids used for peptide synthesis may be standard Boc (N^{a}-amino protected N^{a}-t-butyloxycarbonyl) amino acid resin with the standard deprotecting, neutralization, coupling and wash protocols of the original solid phase procedure of Merrifield (J. Am. Chem. Soc., 85:2149-2154, 1963), or the base-labile N^{a}-amino protected 9-fluorenylmethoxycarbonyl (Fmoc) amino acids first described by Carpino and Han (J. Org. Chem., 37:3403-3409, 1972). Both Fmoc and Boc N^{a}-amino protected amino acids can be obtained from Fluka, Bachem, Advanced Chemtech, Sigma, Cambridge Research Biochemical, Bachem, or Peninsula Labs or other chemical companies familiar to those who practice this art. In addition, the method of the invention can be used with other N^{a}-protecting groups that are familiar to those skilled in this art. Solid phase peptide synthesis may be accomplished by techniques familiar to those in the art and provided, for example, in Stewart and Young, 1984, Solid Phase Synthesis, Second Edition, Pierce Chemical Co., Rockford, Ill.; Fields and Noble, 1990, Int. J. Pept Protein Res. 35:161-214, or using automated synthesizers, such as sold by ABS. Thus, polypeptides of the invention may comprise D-amino acids, a combination of D- and L-amino acids, and various “designer” amino acids (e.g., β-methyl amino acids, Cα-methyl amino acids, and Nα-methyl amino acids, etc.) to convey special properties. Synthetic amino acids include ornithine for lysine, fluorophenylalanine for phenylalanine, and norleucine for leucine or isoleucine. Additionally, by assigning specific amino acids at specific coupling steps, α-helices, β turns, β sheets, γ-turns, and cyclic peptides can be generated.

Also provided in the invention are nucleic acid molecules that selectively hybridize with nucleic acid molecules encoding the collagen-binding proteins of the invention, or portions thereof, such as consensus or variable sequence amino acid motifs, from Staphylococcus epidermidis described herein or complementary sequences thereof. By “selective” or “selectively” is meant a sequence which does not hybridize with other nucleic acids. This is to promote specific detection of GehD. Therefore, in the design of hybridizing nucleic acids, selectivity will depend upon the other components present in a sample. The hybridizing nucleic acid should have at least 70% complementarity with the segment of the nucleic acid to which it hybridizes. As used herein to describe nucleic acids, the term “selectively hybridizes” excludes the occasional randomly hybridizing nucleic acids, and thus, has the same meaning as “specifically hybridizing”. The selectively hybridizing nucleic acids of the invention can have at least 70%, 80%, 85%, 90%, 95%, 97%, 98%, and 99% complementarity with the segment of the sequence to which they hybridize.

The invention contemplates sequences, probes and primers which selectively hybridize to the encoding DNA or the complementary, or opposite, strand of DNA as those specifically provided herein. Specific hybridization with nucleic acid can occur with minor modifications or substitutions in the nucleic acid, so long as functional species-specific hybridization capability is maintained. By “probe” is meant nucleic acid sequences that can be used as probes or primers for selective hybridization with complementary nucleic acid sequences for their detection or amplification, which probes can vary in length from about 5 to 100 nucleotides, or preferably from about 10 to 50 nucleotides, or most preferably about 18-24 nucleotides. Therefore, the terms “probe” or “probes” as used herein are defined to include “primers”. Isolated nucleic acids are provided herein that selectively hybridize with the species-specific nucleic acids under stringent conditions and should have at least 5 nucleotides complementary to the sequence of interest as described by Sambrook et al., 1989. MOLECULAR CLONING: A LABORATORY MANUAL, 2nd ed. Cold Spring Harbor Laboratory, Cold Spring Harbor, N.Y.

If used as primers, the composition preferably includes at least two nucleic acid molecules which hybridize to different regions of the target molecule so as to amplify a desired region. Depending on the length of the probe or primer, the target region can range between 70% complementary bases and full complementarity and still hybridize under stringent conditions. For example, for the purpose of diagnosing the presence of the S. pyogenes, the degree of complementarity between the hybridizing nucleic acid (probe or primer) and the sequence to which it hybridizes (e.g., staphylococcal DNA from a sample) is at least enough to distinguish hybridization with a nucleic acid from other bacteria.

The nucleic acid sequences encoding GehD, such as consensus or variable sequence amino acid motifs, can be inserted into a vector, such as a plasmid, and recombinantly expressed in a living organism to produce recombinant GehD proteins or active fragments thereof.

Recombinant proteins are produced by methods well known to those skilled in the art. For example, a cloning vector, such as a plasmid or phage DNA is cleaved with a restriction enzyme, and the DNA sequence encoding the GehD lipase or active fragments thereof, such as consensus or variable sequence amino acid motifs, is inserted into the cleavage site and ligated. The cloning vector is then inserted into a host to produce the protein or fragment encoded by the GehD-encoding DNA. Suitable hosts include bacterial hosts such as Escherichia coli, Bacillus subtilis, yeasts and other cell cultures. Production and purification of the gene product may be achieved and enhanced using known molecular biology techniques.

In accordance with the invention, a method of diagnosing staphylococcal infections is provided utilizing the collagen-binding GehD lipase of the invention. The methods are useful for diagnosing staphylococcal infections such as may occur in catheter related infections, biomaterial related infections, respiratory tract infections, cardiac, gastrointestinal or central nervous system infections, ocular infections, wound infections, skin infections, and a myriad of other diseases including conjunctivitis, keratitis, cellulitis, myositis, septic arthritis, osteomyelitis, bovine mastitis, and canine pyoderma, all as affected by staphylococcal bacteria.

In accordance with the invention, a preferred method of detecting the presence of GehD proteins involves the steps of obtaining a sample suspected of containing staphylococci. The sample may be taken from an individual, for example, from one's blood, saliva, tissues, bone, muscle, cartilage, or skin. In one method, antibodies raised against GehD will be utilized in a kit wherein the sample can be introduced to the GehD antibodies, and binding of the GehD proteins to the antibodies can be detected using any of a number of conventional labels to determine the presence of GehD proteins. In addition, the cells of the sample can then be lysed, and the DNA extracted, precipitated and amplified. Detection of DNA from staphylococci can then be achieved by hybridizing the amplified DNA with a probe for staphylococcal that selectively hybridizes with the DNA as described above. Detection of hybridization is indicative of the presence of staphylococci.

Preferably, detection of nucleic acid (e.g. probes or primers) hybridization can be facilitated by the use of detectable moieties. For example, the probes can be labeled with biotin and used in a streptavidin-coated microtiter plate assay. Other detectable moieties include radioactive labeling, enzyme labeling, and fluorescent labeling, for example.

DNA may be detected directly or may be amplified enzymatically using polymerase chain reaction (PCR) or other amplification techniques prior to analysis. RNA or cDNA can be similarly detected. Increased or decrease expression of GehD can be measured using any of the methods well known in the art for the quantification of nucleic acid molecules, such as, for example, amplification, PCR, RT-PCR, RNase protection, Northern blotting, and other hybridization methods.

Diagnostic assays for GehD proteins or active portions thereof, such as consensus or variable sequence amino acid motifs, or anti-GehD antibodies may also be used to detect the presence of a staphylococcal bacterium such as Staphylococcus epidermidis. Assay techniques for determining protein or antibody levels in a sample are well known to those skilled in the art and include methods such as radioimmunoasssay, Western blot analysis and ELISA assays.

In accordance with the present invention, isolated and/or purified GehD, either isolated, recombinant or synthetic proteins of the present invention, or antigenic portions thereof (including epitope-bearing fragments), or fusion proteins, can be utilized to generate an immune reaction by administered to humans or animals an immunogenic amount of the isolated GehD or MgehD lipase. In addition, antibodies to GehD in accordance with the invention may be generated via a number of conventional ways, such as by administering an immunogenic amount of GehD or an active fragment thereof to animals as immunogens or antigens, alone or in combination with an adjuvant, which will result in the production of antibodies reactive with GehD proteins or portions thereof which can then be isolated and/or purified in a suitable manner. In addition, the proteins can be used to screen antibodies or antisera for hyperimmune patients from whom can be derived specific antibodies having a very high affinity for the proteins.

Antibodies to GehD, or active fragments thereof, can thus be utilized in accordance with the present invention to treat or prevent a staphylococcal infection in a human or animal patient in need of such treatment. In a preferred method, an effective amount of the antibodies are administered so as to treat or prevent a staphylococcal infection, and such antibodies may also be administered in therapeutic compositions with a suitable vehicle, carrier or excipient. The antibodies in accordance with the invention can also be used for the specific detection of collagen-binding proteins, for the prevention of infection from staphylococci, for the treatment of an ongoing infection, or for use as research tools. The term “antibodies” as used herein includes monoclonal, polyclonal, chimeric, single chain, bispecific, simianized, and humanized or primatized antibodies as well as Fab fragments, including the products of an Fab immunoglobulin expression library. Generation of any of these types of antibodies or antibody fragments is well known to those skilled in the art. In the present case, specific polyclonal antiserum against GehD has been generated which reacts with GehD in Western immunoblots and ELISA assays and interferes with GehD binding to collagen. The antiserum can be used for specific agglutination assays to detect bacteria which express GehD on their surface. The antiserum does not cross-react with bacteria which express the fibronectin-binding protein F1 on their surface, although a portion of protein F1 exhibits sequence homologies to GehD.

Any of the above described antibodies may be labeled directly with a detectable label for identification and quantification of staphylococci. Labels for use in immunoassays are generally known to those skilled in the art and include enzymes, radioisotopes, and fluorescent, luminescent and chromogenic substances, including colored particles such as colloidal gold or latex beads. Suitable immunoassays include enzyme-linked immunosorbent assays (ELISA).

Alternatively, the antibody may be labeled indirectly by reaction with labeled substances that have an affinity for immunoglobulin. The antibody may be conjugated with a second substance and detected with a labeled third substance having an affinity for the second substance conjugated to the antibody. For example, the antibody may be conjugated to biotin and the antibody-biotin conjugate detected using labeled avidin or streptavidin. Similarly, the antibody may be conjugated to a hapten and the antibody-hapten conjugate detected using labeled anti-hapten antibody. These and other methods of labeling antibodies and assay conjugates are well known to those skilled in the art.

Antibodies to the collagen-binding proteins GehD, or portions thereof, may also be used in production facilities or laboratories to isolate additional quantities of the proteins, such as by affinity chromatography. For example, antibodies to the collagen-binding protein GehD may also be used to isolate additional amounts of collagen.

The isolated proteins of the present invention, or active fragments thereof, and antibodies to the proteins may be useful for the treatment and diagnosis of staphylococcal bacterial infections as described above, or for the development of anti-staphylococcal vaccines for active or passive immunization. Further, when administered as pharmaceutical composition to a wound or used to coat medical devices or polymeric biomaterials in vitro and in vivo, both the proteins and the antibodies are useful as blocking agents to prevent or inhibit the binding of staphylococci to the wound site or the biomaterials themselves. Preferably, the antibody is modified so that it is less immunogenic in the patient to whom it is administered. For example, if the patient is a human, the antibody may be “humanized” by transplanting the complementarity determining regions of the hybridoma-derived antibody into a human monoclonal antibody as described, e.g., by Jones et al., Nature 321:522-525 (1986) or Tempest et al. Biotechnology 9:266-273 (1991).

Medical devices or polymeric biomaterials to be coated with the antibodies, proteins and active fragments described herein include, but are not limited to, staples, sutures, replacement heart valves, cardiac assist devices, hard and soft contact lenses, intraocular lens implants (anterior chamber or posterior chamber), other implants such as corneal inlays, kerato-prostheses, vascular stents, epikeratophalia devices, glaucoma shunts, retinal staples, scleral buckles, dental prostheses, thyroplastic devices, laryngoplastic devices, vascular grafts, soft and hard tissue prostheses including, but not limited to, pumps, electrical devices including stimulators and recorders, auditory prostheses, pacemakers, artificial larynx, dental implants, mammary implants, penile implants, cranio/facial tendons, artificial joints, tendons, ligaments, menisci, and disks, artificial bones, artificial organs including artificial pancreas, artificial hearts, artificial limbs, and heart valves; stents, wires, guide wires, intravenous and central venous catheters, laser and balloon angioplasty devices, vascular and heart devices (tubes, catheters, balloons), ventricular assists, blood dialysis components, blood oxygenators, urethral/ureteral/urinary devices (Foley catheters, stents, tubes and balloons), airway catheters (endotracheal and tracheostomy tubes and cuffs), enteral feeding tubes (including nasogastric, intragastric and jejunal tubes), wound drainage tubes, tubes used to drain the body cavities such as the pleural, peritoneal, cranial, and pericardial cavities, blood bags, test tubes, blood collection tubes, vacutainers, syringes, needles, pipettes, pipette tips, and blood tubing.

It will be understood by those skilled in the art that the term “coated” or “coating”, as used herein, means to apply the protein, antibody, or active fragment to a surface of the device, preferably an outer surface that would be exposed to streptococcal bacterial infection. The surface of the device need not be entirely covered by the protein, antibody or active fragment.

In addition, the present invention may be utilized as immunological compositions, including vaccines, and other pharmaceutical compositions containing the GehD proteins or portions thereof are included within the scope of the present invention. A suitable vaccine in accordance with the invention can comprise an immunogenic amount of the GehD lipase, or an active fragment thereof, e.g., MgehD, preferably administered in a suitable vehicle, carrier or excipient, such as any of a number of conventional vehicles, carriers and excipients well known to one of ordinary skill in the art. Thus, the GehD lipase of the invention, or active or antigenic fragments thereof, or fusion proteins thereof, can be formulated and packaged, alone or in combination with other antigens, using methods and materials known to those skilled in the art for vaccines. The immunological response may be used therapeutically or prophylactically and may provide antibody immunity or cellular immunity, such as that produced by T lymphocytes.

The immunological compositions, such as vaccines, and other pharmaceutical compositions can be used alone or in combination with other blocking agents to protect against human and animal infections caused by or exacerbated by staphylococci. For example, the compositions may be effective against a variety of conditions, including use to protect humans against skin infections such as impetigo and eczema, as well as mucous membrane infections such as tonsillopharyngitis. In addition, effective amounts of the compositions of the present invention may be used to protect against complications caused by localized infections such as sinusitis, mastoiditis, parapharygeal abscesses, cellulitis, necrotizing fascitis, myositis, streptococcal toxic shock syndrome, pneumonitis endocarditis, meningitis, osteomylitis, and many other sever diseases. Further, the present compositions can be used to protect against nonsuppurative conditions such as acute rheumatic fever, acute glomerulonephritis, obsessive/compulsive neurologic disorders and exacerbations of forms of psoriasis such as psoriasis vulgaris. The compositions may also be useful as appropriate in protecting both humans and other species of animals where needed to combat similar staphylococcal infections.

To enhance immunogenicity, the proteins may be conjugated to a carrier molecule. Suitable immunogenic carriers include proteins, polypeptides or peptides such as albumin, hemocyanin, thyroglobulin and derivatives thereof, particularly bovine serum albumin (BSA) and keyhole limpet hemocyanin (KLH), polysaccharides, carbohydrates, polymers, and solid phases. Other protein derived or non-protein derived substances are known to those skilled in the art. An immunogenic carrier typically has a molecular weight of at least 1,000 Daltons, preferably greater than 10,000 Daltons. Carrier molecules often contain a reactive group to facilitate covalent conjugation to the hapten. The carboxylic acid group or amine group of amino acids or the sugar groups of glycoproteins are often used in this manner. Carriers lacking such groups can often be reacted with an appropriate chemical to produce them. Preferably, an immune response is produced when the immunogen is injected into animals such as mice, rabbits, rats, goats, sheep, guinea pigs, chickens, and other animals, most preferably mice and rabbits. Alternatively, a multiple antigenic peptide comprising multiple copies of the protein or polypeptide, or an antigenically or immunologically equivalent polypeptide may be sufficiently antigenic to improve immunogenicity without the use of a carrier.

The GehD protein, or active portions thereof, or combination of proteins, may be administered with an adjuvant in an amount effective to enhance the immunogenic response against the conjugate. For example, an adjuvant widely used in humans has been alum (aluminum phosphate or aluminum hydroxide). Saponin and its purified component Quil A, Freund's complete adjuvant and other adjuvants used in research and veterinary applications have toxicities which limit their potential use in human vaccines. However, chemically defined preparations such as muramyl dipeptide, monophosphoryl lipid A, phospholipid conjugates such as those described by Goodman-Snitkoff et al. J. Immunol. 147:410-415 (1991) and incorporated by reference herein, encapsulation of the conjugate within a proteoliposome as described by Miller et al., J. Exp. Med. 176:1739-1744 (1992) and incorporated by reference herein, and encapsulation of the protein in lipid vesicles such as Novasome™ lipid vesicles (Micro Vescular Systems, Inc., Nashua, N.H.) may also be useful.

The term “vaccine” as used herein includes not only vaccines comprising GehD proteins but of nucleic acids coding for the GehD lipase which may also be used in a pharmaceutical composition that may be administered to a patient. For genetic immunization, suitable delivery methods known to those skilled in the art include direct injection of plasmid DNA into muscles (Wolff et al., Hum. Mol. Genet. 1:363, 1992), delivery of DNA complexed with specific protein carriers (Wu et al., J. Biol. Chem. 264:16985, 1989), coprecipitation of DNA with calcium phosphate (Benvenisty and Reshef, Proc. Natl. Acad. Sci. 83:9551, 1986), encapsulation of DNA in liposomes (Kaneda et al., Science 243:375, 1989), particle bombardment (Tang et al., Nature 356:152, 1992 and Eisenbraun et al., DNA Cell Biol. 12:791, 1993), and in vivo infection using cloned retroviral vectors (Seeger et al., Proc. Natl. Acad. Sci. 81:5849, 1984).

There are several advantages of immunization with a gene rather than its gene product. The first is the relative simplicity with which native or nearly native antigen can be presented to the immune system. Mammalian proteins expressed recombinantly in bacteria, yeast, or even mammalian cells often require extensive treatment to ensure appropriate antigenicity. A second advantage of DNA immunization is the potential for the immunogen to enter the MHC class I pathway and evoke a cytotoxic T cell response. Immunization of mice with DNA encoding the influenza A nucleoprotein (NP) elicited a CD8⁺ response to NP that protected mice against challenge with heterologous strains of flu. (See Montgomery, D. L. et al., Cell Mol Biol, 43(3):285-92, 1997 and Ulmer, J. et al., Vaccine, 15(8):792-794, 1997.)

Cell-mediated immunity is important in controlling infection. Since DNA immunization can evoke both humoral and cell-mediated immune responses, its greatest advantage may be that it provides a relatively simple method to survey a large number of S. pyogenes genes for their vaccine potential.

Pharmaceutical compositions containing the GehD lipase or active portions thereof such as MGehD, nucleic acid molecules, antibodies, or fragments thereof, may be formulated in combination with a pharmaceutical excipient or carrier such as saline, dextrose, water, glycerol, ethanol, other therapeutic compounds, and combinations thereof. The formulation should be appropriate for the mode of administration. The compositions are useful for interfering with, modulating, or inhibiting binding interactions between streptococcal bacteria and collagen on host cells.

The amount of expressible DNA or transcribed RNA to be introduced into a vaccine recipient will have a very broad dosage range and may depend on the strength of the transcriptional and translational promoters used. In addition, the magnitude of the immune response may depend on the level of protein expression and on the immunogenicity of the expressed gene product. In general, effective dose ranges of about 1 ng to 5 mg, 100 ng to 2.5 mg, 1 μg to 750 μg, and preferably about 10 μg to 300 μg of DNA is administered directly into muscle tissue. Subcutaneous injection, intradermal introduction, impression through the skin, and other modes of administration such as intraperitoneal, intravenous, or inhalation delivery are also suitable. It is also contemplated that booster vaccinations may be provided. Following vaccination with a polynucleotide immunogen, boosting with protein immunogens such as the GehD gene product is also contemplated. For administration of compounds in accordance with the invention, an “effective amount” is that amount that would be readily determinable by one of ordinary skill in the art to effectively treat a patient or to prevent infection in a patient, and that amount would be determined by the specific circumstances and conditions surrounding the mode of therapy, such as nature of patient and condition to be treated, nature of the materials used in the composition, state of the infection, desired purpose for the treatment, etc.

If using a polynucleotide coding the proteins of the invention in administerable compositions, the polynucleotide may be “naked”, that is, unassociated with any proteins, adjuvants or other agents which affect the recipient's immune system. In this case, it is desirable for the polynucleotide to be in a physiologically acceptable solution, such as, but not limited to, sterile saline or sterile buffered saline. Alternatively, the DNA may be associated with liposomes, such as lecithin liposomes or other liposomes known in the art, as a DNA-liposome mixture, or the DNA may be associated with an adjuvant known in the art to boost immune responses, such as a protein or other carrier. Agents which assist in the cellular uptake of DNA, such as, but not limited to, calcium ions, may also be used. These agents are generally referred to herein as transfection facilitating reagents and pharmaceutically acceptable carriers. Techniques for coating microprojectiles coated with polynucleotide are known in the art and are also useful in connection with this invention. For DNA intended for human use it may be useful to have the final DNA product in a pharmaceutically acceptable carrier or buffer solution. Pharmaceutically acceptable carriers or buffer solutions are known in the art and include those described in a variety of texts such as Remington's Pharmaceutical Sciences.

It is recognized by those skilled in the art that an optimal dosing schedule for a DNA vaccination regimen may include as many as five to six, but preferably three to five, or even more preferably one to three administrations of the immunizing entity given at intervals of as few as two to four weeks, to as long as five to ten years, or occasionally at even longer intervals.

Suitable methods of administration of any pharmaceutical composition disclosed in this application include, but are not limited to, topical, oral, anal, vaginal, intravenous, intraperitoneal, intramuscular, subcutaneous, intranasal and intradermal administration.

For topical administration, the composition is formulated in the form of an ointment, cream, gel, lotion, drops (such as eye drops and ear drops), or solution (such as mouthwash). Wound or surgical dressings, sutures and aerosols may be impregnated with the composition. The composition may contain conventional additives, such as preservatives, solvents to promote penetration, and emollients. Topical formulations may also contain conventional carriers such as cream or ointment bases, ethanol, or oleyl alcohol.

In a preferred embodiment, a vaccine is packaged in a single dosage for immunization by parenteral (i.e., intramuscular, intradermal or subcutaneous) administration or nasopharyngeal (i.e., intranasal) administration. The vaccine is most preferably injected intramuscularly into the deltoid muscle. The vaccine is preferably combined with a pharmaceutically acceptable carrier to facilitate administration. The carrier is usually water or a buffered saline, with or without a preservative. The vaccine may be lyophilized for resuspension at the time of administration or in solution.

Microencapsulation of the protein will give a controlled release. A number of factors contribute to the selection of a particular polymer for microencapsulation. The reproducibility of polymer synthesis and the microencapsulation process, the cost of the microencapsulation materials and process, the toxicological profile, the requirements for variable release kinetics and the physicochemical compatibility of the polymer and the antigens are all factors that must be considered. Examples of useful polymers are polycarbonates, polyesters, polyurethanes, polyorthoesters, polyamides, poly (D,L-lactide-co-glycolide) (PLGA) and other biodegradable polymers. The use of PLGA for the controlled release of antigen is reviewed by Eldridge et al., CURRENT TOPICS IN MICROBIOLOGY AND IMMUNOLOGY, 146:59-66 (1989).

The preferred dose for human administration is from 0.01 mg/kg to 10 mg/kg, preferably approximately 1 mg/kg. Based on this range, equivalent dosages for heavier body weights can be determined. The dose should be adjusted to suit the individual to whom the composition is administered and will vary with age, weight and metabolism of the individual. The vaccine may additionally contain stabilizers or pharmaceutically acceptable preservatives, such as thimerosal (ethyl(2-mercaptobenzoate-S)mercury sodium salt) (Sigma Chemical Company, St. Louis, Mo.).

When labeled with a detectable biomolecule or chemical, the collagen-binding proteins described herein are useful for purposes such as in vivo and in vitro diagnosis of streptococcal infections or detection of staphylococcal bacteria. Laboratory research may also be facilitated through use of such protein-label conjugates. Various types of labels and methods of conjugating the labels to the proteins are well known to those skilled in the art. Several specific labels are set forth below. The labels are particularly useful when conjugated to a protein such as an antibody or receptor. For example, the protein can be conjugated to a radiolabel such as, but not restricted to, ³²P, ³H, ¹⁴C, ³⁵S, ¹²⁵I, or ¹³¹I. Detection of a label can be by methods such as scintillation counting, gamma ray spectrometry or autoradiography.

Bioluminescent labels, such as derivatives of firefly luciferin, are also useful. The bioluminescent substance is covalently bound to the protein by conventional methods, and the labeled protein is detected when an enzyme, such as luciferase, catalyzes a reaction with ATP causing the bioluminescent molecule to emit photons of light. Fluorogens may also be used to label proteins. Examples of fluorogens include fluorescein and derivatives, phycoerythrin, allo-phycocyanin, phycocyanin, rhodamine, and Texas Red. The fluorogens are generally detected by a fluorescence detector.

The protein can alternatively be labeled with a chromogen to provide an enzyme or affinity label. For example, the protein can be biotinylated so that it can be utilized in a biotin-avidin reaction, which may also be coupled to a label such as an enzyme or fluorogen. For example, the protein can be labeled with peroxidase, alkaline phosphatase or other enzymes giving a chromogenic or fluorogenic reaction upon addition of substrate. Additives such as 5-amino-2,3-dihydro-1,4-phthalazinedione (also known as Luminol^{a}) (Sigma Chemical Company, St. Louis, Mo.) and rate enhancers such as p-hydroxybiphenyl (also known as p-phenylphenol) (Sigma Chemical Company, St. Louis, Mo.) can be used to amplify enzymes such as horseradish peroxidase through a luminescent reaction; and luminogeneic or fluorogenic dioxetane derivatives of enzyme substrates can also be used. Such labels can be detected using enzyme-linked immunoassays (ELISA) or by detecting a color change with the aid of a spectrophotometer. In addition, proteins may be labeled with colloidal gold for use in immunoelectron microscopy in accordance with methods well known to those skilled in the art.

The location of a ligand in cells can be determined by labeling an antibody as described above and detecting the label in accordance with methods well known to those skilled in the art, such as immunofluorescence microscopy using procedures such as those described by Warren and Nelson (Mol. Cell. Biol., 7: 1326-1337, 1987).

In addition to the therapeutic compositions and methods described above, the GehD proteins or active portions or fragments thereof, nucleic acid molecules or antibodies are useful for interfering with the initial physical interaction between a pathogen and mammalian host responsible for infection, such as the adhesion of bacteria, to mammalian extracellular matrix proteins such as collagen on in-dwelling devices or to extracellular matrix proteins in wounds; to block GehD protein-mediated mammalian cell invasion; to block bacterial adhesion between collagen and bacterial GehD proteins or portions thereof that mediate tissue damage; and, to block the normal progression of pathogenesis in infections initiated other than by the implantation of in-dwelling devices or surgical techniques.

The GehD proteins, or active fragments thereof, are useful in a method for screening compounds to identify compounds that inhibit collagen binding of streptococci to host molecules. In accordance with the method, the compound of interest is combined with one or more of the GehD proteins or fragments thereof and the degree of binding of the protein to collagen or other extracellular matrix proteins is measured or observed. If the presence of the compound results in the inhibition of protein-collagen binding, for example, then the compound may be useful for inhibiting staphylococci in vivo or in vitro. The method could similarly be used to identify compounds that promote interactions of staphylococcal with host molecules. The method is particularly useful for identifying compounds having bacteriostatic or bacteriocidal properties.

For example, to screen for staphylococcal agonists or antagonists, a synthetic reaction mixture, a cellular compartment (such as a membrane, cell envelope or cell wall) containing one or more of the GehD proteins or fragments thereof and a labeled substrate or ligand of the protein is incubated in the absence or the presence of a compound under investigation. The ability of the compound to agonize or antagonize the protein is shown by a decrease in the binding of the labeled ligand or decreased production of substrate product. Compounds that bind well and increase the rate of product formation from substrate are agonists. Detection of the rate or level of production of product from substrate may be enhanced by use of a reporter system, such as a calorimetric labeled substrate converted to product, a reporter gene that is responsive to changes in GehD nucleic acid or protein activity, and binding assays known to those skilled in the art. Competitive inhibition assays can also be used.

Potential antagonists include small organic molecules, peptides, polypeptides and antibodies that bind to GehD nucleic acid molecules or proteins or portions thereof and thereby inhibit their activity or bind to a binding molecule (such as collagen to prevent the binding of the GehD nucleic acid molecules or proteins to its ligand. For example, a compound that inhibits GehD activity may be a small molecule that binds to and occupies the binding site of the GehD protein, thereby preventing binding to cellular binding molecules, to prevent normal biological activity. Examples of small molecules include, but are not limited to, small organic molecule, peptides or peptide-like molecules. Other potential antagonists include antisense molecules. Preferred antagonists include compounds related to and variants or derivatives of the GehD proteins or portions thereof. The nucleic acid molecules described herein may also be used to screen compounds for antibacterial activity.

The invention further contemplates a kit containing one or more GehD-specific nucleic acid probes, which can be used for the detection of collagen-binding proteins from staphylococci in a sample, or for the diagnosis of staphylococcal bacterial infections. Such a kit can also contain the appropriate reagents for hybridizing the probe to the sample and detecting bound probe. In an alternative embodiment, the kit contains antibodies specific to either or both GehD proteins or active portions thereof which can be used for the detection of staphylococci.

In yet another embodiment, the kit contains a GehD lipase, or active fragments thereof such as MGehD, which can be used for the detection of staphylococcal bacteria or for the presence of antibodies to collagen-binding staphylococcal proteins in a sample. The kits described herein may additionally contain equipment for safely obtaining the sample, a vessel for containing the reagents, a timing means, a buffer for diluting the sample, and a calorimeter, reflectometer, or standard against which a color change may be measured.

In a preferred embodiment, the reagents, including the protein or antibody, are lyophilized, most preferably in a single vessel. Addition of aqueous sample to the vessel results in solubilization of the lyophilized reagents, causing them to react. Most preferably, the reagents are sequentially lyophilized in a single container, in accordance with methods well known to those skilled in the art that minimize reaction by the reagents prior to addition of the sample.

Still other features, uses and advantages of the invention will be obtained as described for other collagen binding proteins, such as those set forth in U.S. Pat. Nos. 5,851,794 and 6,288,214, incorporated herein by reference.

While the invention has been described above with regard to preferred embodiments, it is clear to one skilled in the art that there will be additional embodiments, compositions and methods which fall within the scope of the invention which have not been specifically described above.

The following examples are provided which exemplify aspects of the preferred embodiments of the present invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE

### Determining the Collagen Binding Properties of GehD Lipase, and Generation of Antibodies Thereto

Experimental Procedures.

Bacterial Strains and Culture Conditions.

S. epidermidis strains 146, 9491, 12228, 14852 and 14990 were obtained from the ATCC collection. S. epidermidis 9, 2J24 (gehC::ermC), and KIC82 (gehD::ermC) were created by Christopher M. Longshaw (12). S. aureus Cowan 1 □ispa::TcR strain was generously donated by T. Foster (University of Dublin, Ireland). All strains were grown in brain heart infusion (BHI) or triptic soy broth (TSB) media (Difco, Detroit, Mich.) at 37° C. overnight. For the monoclonal antibody reactivity assays, bacteria were harvested and re-suspended in 10 mM sodium phosphate buffer, pH 7.4, containing 0.13M NaCl (phosphate-buffered saline, PBS) and 0.02% sodium azide, washed, and adjusted to a cell density of 10¹⁰ cells/ml using a standard curve relating the OD₆₀₀ to the cell number determined counting cells in a Petroff-Hausser chamber. The cells were then heat-killed at 88° C. for 10 minutes. For all other assays, overnight cultures were diluted 1:1000 into fresh TSB media, and the resultant culture was incubated until it reached logarithmic growth phase (OD₆₀₀ 0.3-0.6). Bacteria was then harvested by centrifugation and used in attachment or Western assays.

Library Construction.

A S. epidermidis 9491 λZAP Express (Stratagene) expression library was constructed as follows. S. epidermidis 9491 chromosomal DNA was partially digested with Mbol, and the fragments corresponding to 3-11 kb were isolated and purified. The purified fragments were ligated to the ZAP Express® (Stratagene) vector, predigested with BamHI and dephosphorylated with CIAP. The resultant ligation product was packaged into phage particles using the Gigapack III Gold (Stratagene) packaging extract. The obtained library was amplified and screened using the E. coli XL1-Blue MRF' strain. Clones of interest were excised from the λ ZAP Express® phage using the ExAssist® helper phage to generate the pBK-CMV phagemid vector packaged as filamentous phage particles. The filamentous phage stock was used to infect the E. coli XLOLR strain. The resultant colonies carrying the excised pBK-CMV phagemid vector were used for subsequent subcloning and dideoxy-sequencing of the cloned inserts.

A DNA fragment encoding the mature domain of the GehD lipase was PCR amplified from S. epidermidis 9491 genomic DNA. The oligonucleotides primers 5′ TTT GAA TTC TGC GCA AGC TCA ATA TAA and 5′ TTT GCG GCC GCT ATC GCT ACT TAC GTG TAA were used to amplify the fragment designated a MGehD. Constructs generated by PCR were cloned into the pETBlue-2 System, using the E. coli NovaBlue strain as a cloning host, and the E. coli Tuner (DE3) pLacl strain as the expression host.

Large-scale expression and preparation of recombinant proteins were as described previously using HiTrap Ni chelating chromatography (1). Protein concentrations were determined from the absorbance at 280 nm as measured on a Beckman Du-70 UV-visible spectrophotometer. The molar extinction coefficient of the proteins was calculated using the method of Pace et al. (16).

Labeling of Proteins.

Purified collagen I (Vitrogen®, Cohesion, Palo Alto Calif.) was labeled with digoxigenin-3-O-methylcarbonyl-e-aminocaproic acid-N-hydroxy-succinimide ester (digoxigenin) (Boehringer Mannheim) according to the manufacturer's instructions.

To label recombinant proteins with biotin, 7.5 mg of NHS-LC-biotin [sulphosuccinimidyl-6-(biotinamido) hexanoate; Pierce] was dissolved in 100 μl of DMSO and combined with 0.5 mg of recombinant protein in PBS. The total reaction (1 ml volume) was incubated on an end-over-end rotator at room temperature for 2 h, then dialyzed against PBS and stored at 4° C.

Library Screens.

Digoxigenin-labeled collagen or mAbs 11H11 and 1F6 were used to screen the S. epidermidis 9491 λZAP Express (Stratagene) expression library. The library was plated using standard methods according to the vector manufacturer's instructions (Stratagene). After blocking additional protein-binding sites on the filter lifts with a solution containing 3% bovine serum albumin (BSA), digoxigenin-labeled collagen was allowed to bind to proteins on the filter, followed by binding of anti-digoxigenin F_{ab} conjugated to alkaline phosphatase (Boehringer Mannheim) to the digoxigenin-labeled collagen. When mAbs 11H11 or 1F6 were allowed to bind to proteins on the membranes, goat anti-mouse antibodies conjugated to alkaline phosphatase (Bio-Rad) were used as secondary antibodies. Clones expressing collagen-binding proteins were identified developing the membranes with 5-bromo-4-chlor-3-indoyl phosphate p-toluidine salt (BCIP) and p-nitroblue tetrazolium chloride (NBT) (Bio-Rad).

Enzyme-Linked Immunosorbent Assay (ELISA).

To test the reactivity of the mAbs generated against CNA(151-318) to S. epidermidis strains, microtiter wells (Dasit, Milan, Italy) were incubated overnight at 40° C. with 100 μl of 50 mM sodium carbonate, pH 9.5, containing 20 μg of human fibronectin (HFn) per ml. The wells were then washed five times with 10 mM sodium phosphate buffer, pH 7.4, containing 0.13M NaCl and 0,1% Tween 20 (PBST). Additional protein binding sites in the wells were blocked by incubation for 1 hour with 200 μl of 2% (wt/vol) bovine serum albumin (BSA) in PBS. The wells were incubated for 2 hours at 22° C. with 100 μl of 2% BSA containing 1×10⁸ cells of S. epidermidis or S. aureus Cowan 1 Δspa::Tc^{R}. Unbound cells were removed by washing the wells five times with PBS. Bound cells were incubated for 2 hours at 22° C. with 2 μg of the indicated monoclonal antibodies dissolved in 100 μl of 2% BSA in PBS. After extensive washing with PBST, bound antibody was detected by incubation for 1 hour at 22° C. with peroxidase-conjugated rabbit anti-mouse IgG (Dako, Gostrup, Denmark) diluted 1:500. After being washed, the conjugated enzyme was reacted with o-phenylenediamine dihydrochloride (Sigma), and the absorbance at 492 nm was monitored with a microplate reader (Bio-Rad).

To test protein-protein interactions, enzyme-linked immunosorbent assay (ELISA) plates were coated with 1 μg of type I collagen in 100 μl of PBS per well overnight at 4° C. Wells were then washed three times with PBS and blocked with 1% bovine serum albumin in PBS for 1 h before the addition of varying concentrations of the biotinylated recombinant protein. After incubation at room temperature for 2 h with gentle shaking, the wells were extensively washed with PBS containing 0.05% Tween 20 (PBST). Streptavidin-alkaline phosphatase (AP) conjugate (Boehringer Manheim, Indianapolis, Ind.) was diluted 10,000 fold with blocking buffer and added to the wells. After incubation at room temperature for 45 min, the wells were washed with PBST. For color development, 100 μl of 1.3 M diethanolamine (DEA), pH 9.8, containing 1 mg/ml p-nitrophenyl phosphate (pNPP) (Sigma) was added to the wells. Absorbance at 405 nm (A 405 nm) was measured using a Thermomax microplate reader (Molecular Devices Corporation, Menlo Park, Calif.) after one hour of incubation at room temperature. Experiments were performed in triplicate and repeated with independently prepared protein preparations. Binding to BSA coated wells was considered as background level and subtracted from binding to collagen. Data were presented as the mean value±standard error of A₄₀₅nm from a representative experiment (n=3).

Competition ELISAs were performed as described above except that biotinylated proteins were mixed with antibodies at varying ratios and added to the wells.

Preparation of Polyclonal Antibodies.

Purified mature GehD was dialyzed against 1-mM Na2HPO4, 150 mM NaCl, pH 7.4 (PBS), before being sent to Rockland Immunochemicals, Inc. (Gilbertsville, Pa.), for immunization in rabbits and production of polyclonal antisera. IgGs were purified from both immune and pre-immune serum by chromatography using Protein A-Sepharose (Sigma).

Bacterial Adherence Assays.

Enzyme-linked immunosorbent assay (ELISA) plates were coated with 1 μg of type I collagen in 100 μl of PBS per well overnight at 4° C. Wells were then washed three times with PBS and then blocked with 1% bovine serum albumin in PBS for 1 h before the addition of bacteria. Early log-phase S. epidermidis cultures (OD600 of 0.5) were added and the plates incubated for 2 h. at room temperature. After gentle washes by hand, adherent cells were fixed with 100 μl of 25% aqueous formaldehyde and incubated at room temperature for at least 30 min. The plates were then washed gently, stained with crystal violet, then washed again and read on an ELISA plate reader at 590 nm.

To study inhibition of collagen binding by IgGs, S. epidermidis suspensions were pre-incubated with serial dilutions of purified IgGs in PBS for 2 h at room temperature. The cell suspensions were then transferred to ELISA plates coated with 1 μg of collagen per well and their ability to attach to collagen was tested as described above.

SDS-PAGE and Western Ligand Blot.

Recombinant and native proteins were fractionated by SDS-PAGE (10) and, in Western assays, probed using digoxigenin-labeled collagen or antibodies. For whole-cells SDS-PAGE, 2×10⁷ S. epidermidis (previously treated with lysostaphin) cells or E. coli cells were boiled in sodium dodecyl sulfate (SDS) for 3-5 min under reducing conditions and subjected to electrophoresis through a 10% acrylamide gel at 150V for 45 min. The separated proteins were stained with coomassie brilliant blue.

For Western ligand blot assays, whole cell lysates or purified proteins were transferred from the polyacrylamide gel onto a nitrocellulose membrane in a semi-dry electroblot system (Bio-Rad). Additional binding sites on the membrane were blocked by incubating it in 2% BSA in TBST (0.15 M NaCl, 20 mM Tris-HCl, 0.05% Tween 20, pH 7.4) for 2 h at room temperature or overnight at 4° C., followed by three 10 min washes in TBST. The membrane was then incubated at room temperature with 0.5 μg of digoxigenin-labelled collagen per ml TBST for 1 h, washed and incubated with 1:5000 anti-digoxigenin Fab alkaline-phosphatase conjugate (Boehringer Mannheim) in TBST for 1 h. The membrane was washed, and collagen-binding proteins were visualized with 150 μg 5-bromo-4-chlor-3-indoyl phosphate p-toluidine salt (BCIP) per ml and 300 μg p-nitroblue tetrazolium chloride (NBT) per ml (Bio-Rad) in carbonate bicarbonate buffer (14 mM Na₂CO₃, 36 mM NaHCO₃, 5 mM MgCl₂.6H₂O, pH 9.8).

Results:

Adherence of S. epidermidis 9491 to Extracellular Matrix Proteins.

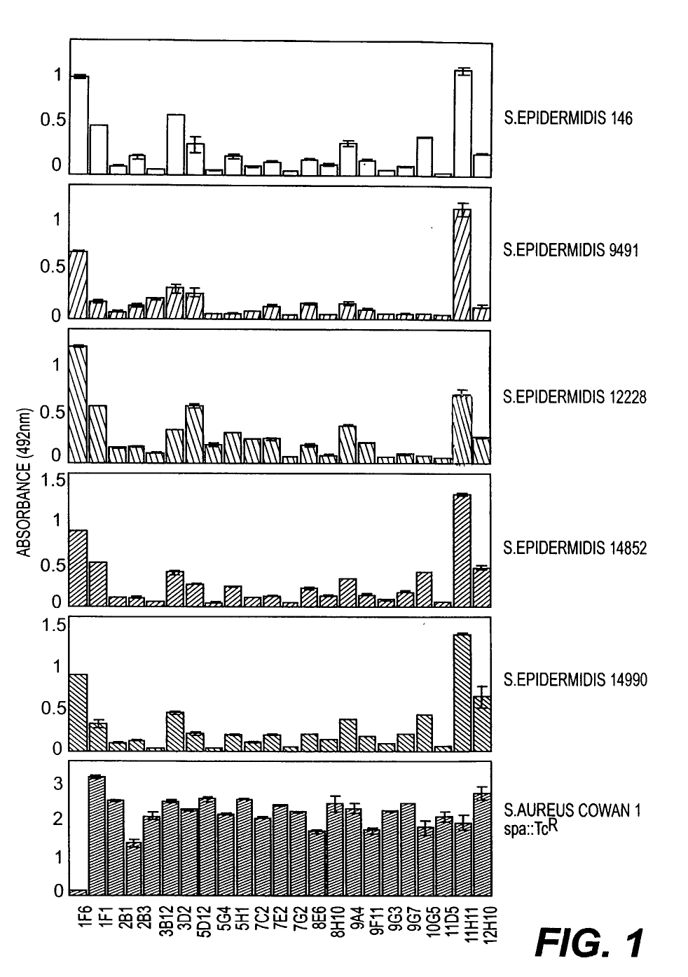

The clinical isolate S. epidermidis 9491 was chosen as a prototype strain in our search for new MSCRAMMs. We tested its ability to bind to immobilized collagen, human fibrinogen and human fibronectin. Each protein was immobilized in microtiter wells and the bacteria attached to the wells were detected using crystal violet. The results presented in FIG. 1 show that S. epidermidis 9491 has the ability to attach to collagen, human fibrinogen and human fibronectin. While it is recognized that S. epidermidis attachment to HFg is mediated by proteins such as Fbe and SdrG, the proteins that mediate attachment to collagen or fibronectin were, up to this point, not determined.

Binding of Monoclonal Antibodies to S. epidermidis Strains.

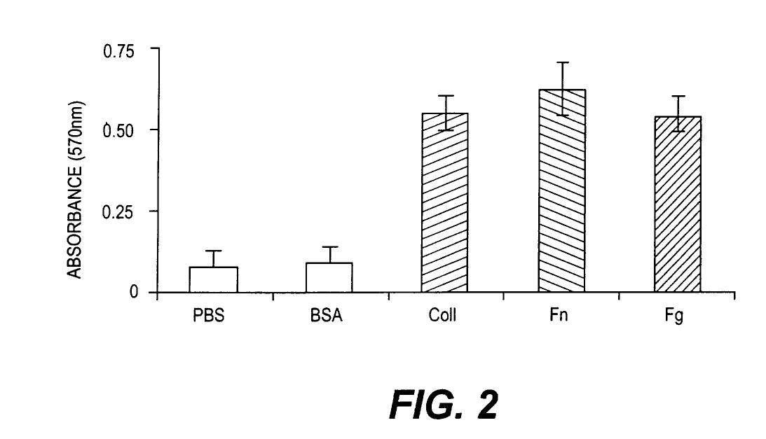

A panel of 22 monoclonal antibodies was previously generated against the Staphylococcus aureus adhesin Cna (151-318) (22). We reasoned that we could use these mAbs as tools to search for collagen-binding proteins in S. epidermidis strains (FIG. 2). At least two monoclonals, 11H11 and 1F6, cross-reacted with whole cells of all the S. epidermidis strains tested. Both of these antibodies were determined to bind to the central region of Cna(151-318). These antibodies recognize conformationally dependent epitopes, since none of the mAbs reacted to synthesized linear peptides that spanned the entire Cna(151-318) sequence (22). These results suggest that S. epidermidis exposes on its surface epitopes similar to those present on Cna, and that these conformationally-dependent epitopes are recognized by 1F6 and 11H11.

Construction of an Expression Library and Identification of a New Collagen-Binding Protein.

We constructed an expression library ligating Mbol-partially digested, size-selected genomic DNA from S. epidermidis 9491 to BamHI-digested λZAP Express II® vector. Using mAbs 1F6 and 11H11, as well as digoxigenin-labeled collagen, we screened approximately 690,000 plaques with each mAb and labeled collagen, and isolated three clones. DNA sequencing of the excised phagemids revealed that two of the clones were identical, and the third had an additional 36 bps of upstream sequence. Further analysis revealed that the cloned DNA immediately downstream of the T7lac sequence from the phagemid is 97% identical to the S. epidermidis second lipase gene, gehD (12).

Purification and Characterization of Recombinant, Mature GehD.

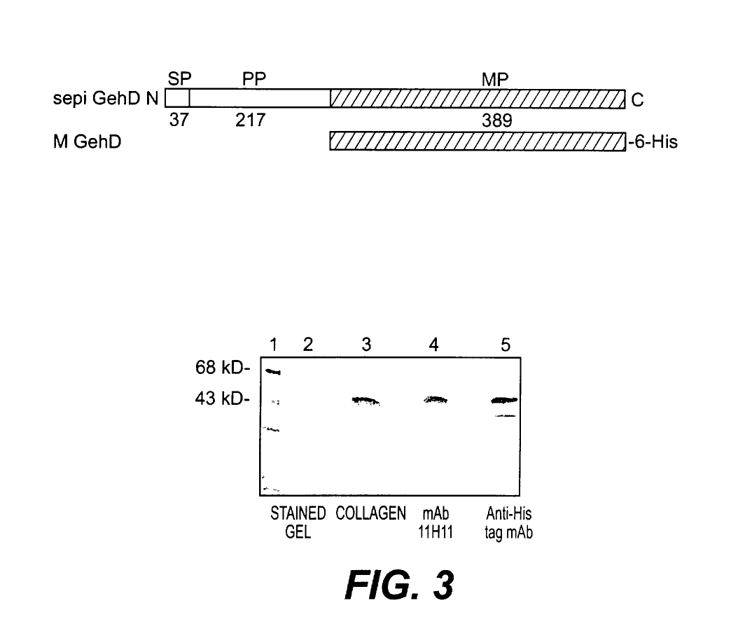

Previous studies of GehD and other staphylococcal lipases have shown that they are transcribed and translocated as 650-700 amino acid precursors that are processed post-translationally to extracellular mature lipases of about 360 aminoacids with a size of approximately 45 kDa (12). To simulate the native protein in the mature form, we used the polymerase chain reaction (PCR) to construct recombinant mature GehD. The protein was expressed as a C-terminal polyhistidine (His-tag) fusion and purified by nickel-chelating chromatography. Mature GehD (MGehD) appears as a single band at approximately 45 kDa when analyzed by SDS-PAGE (FIG. 3).

Amino acid sequence comparisons did not reveal any significant similarities between the linear amino acid sequences of Cna and MGehD. Furthermore, CD spectroscopy deconvolution analysis revealed that the predicted overall secondary structure of MGehD consists of approximately 26.5% α-helix, 20.6% β-sheet and 52.9% coil. This secondary structure composition differs markedly from that of the reported crystal structure of Cna (151-318): 8% α-helix, 53% β-sheet, and 39% coil (19). This data suggest that these proteins, although being recognized by common antibodies, may have radically different secondary structures.

Recombinant MGehD Binds to Collagen.

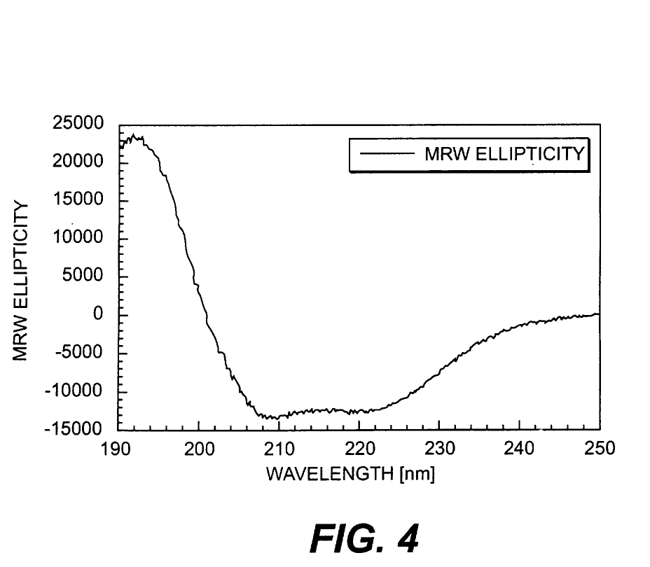

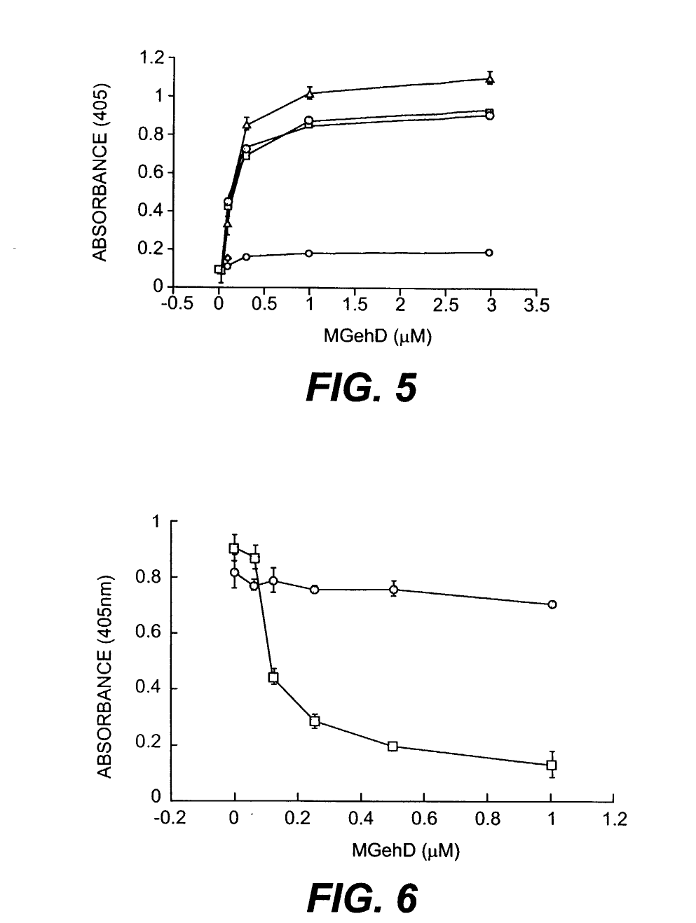

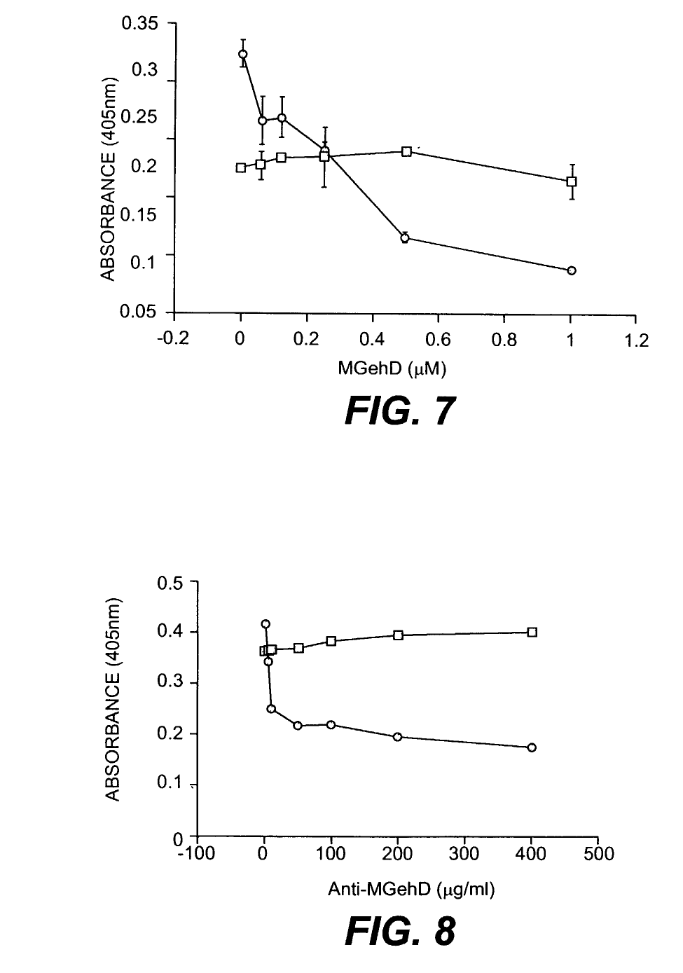

The collagen-binding activity of the recombinant, mature GehD was analyzed by Western ligand blot. Purified protein was separated by SDS-PAGE, transferred to a nitrocellulose membrane and incubated with digoxigenin-labeled collagen, or mAbs 11H11 (anti Cna) or 7E8 (anti His-tag) (FIG. 3). The recombinant, mature GehD binds to collagen and to both antibodies. The collagen-binding activity of the recombinant, biotin-labeled MGehD was also assessed by ELISA. MGehD bound in a concentration-dependent, saturable manner to collagens I, II and IV coated on microtiter wells (FIG. 4). This suggests that recombinant MGehD can bind to immobilized collagen and that the affinity of this interaction is similar to that of Cna.

MGehD Can Inhibit the Attachment of S. epidermidis to Collagen.

We used a microtiter well attachment assay to study the adherence of S. epidermidis to collagen. The effects of purified, recombinant mature GehD on bacterial adherence were examined in experiments in which collagen-coated microtiter wells were pre-incubated with increasing concentrations of recombinant MGehD for 1 hour before whole S. epidermidis were added. Purified MGehD can effectively block the attachment of S. epidermidis 9491, but it does not affect the already decreased attachment of a gehD null strain.

Antibodies Generated Against MGehD Can Block the Attachment of S. epidermidis to Collagen.

We generated polyclonal antibodies against the recombinant, mature GehD protein and assessed their specificity in ELISAs. Purified anti-MGehD IgGs effectively inhibit the binding of biotin-labeled MGehD to immobilized collagen, whereas purified, pre-immune IgGs had no noticeable effect. In addition, we tested the specificity of the antisera using E. coli and S. epidermidis cell extracts. The anti-MGehD purified IgGs recognizes a protein band of approximately 45 kDa in both E. coli expressing the gehD gene or S. epidermidis. This 45-kDa band is not present in the gehD mutant cell lysates. This data shows that anti-GehD IgGs are specific for MGehD.

We therefore used these antibodies in a microtiter well attachment assay to test their ability to inhibit the attachment of whole S. epidermidis cells to immobilized collagen. S. epidermidis cells were pre-incubated with increasing concentrations of purified anti-MGehD antibodies before the cell suspensions were added to collagen-coated microtiter wells. Attached cells were detected using crystal violet. Purified, anti-MGehD antibodies effectively inhibit the attachment of S. epidermidis to collagen. Pre-immune purified IgGs had no noticeable effect (not shown). The same purified IgGs do not seem to affect the already decreased attachment of the gehD null strain. These data suggest that surface-exposed MGehD may mediate the attachment of S. epidermidis to collagen-coated surfaces.

Discussion.

The GehD lipase had been previously identified and characterized as the second glycerol ester hydrolase of Staphylococcus epidermidis (12). The tests conducted in accordance with the invention show that, in addition to its lipolytic activity, GehD can bind to collagen. We show that the recombinant, mature GehD can bind to immobilized or soluble collagen, and that it can act as an inhibitor of S. epidermidis attachment to immobilized collagen. Furthermore, we show that anti-GehD antibodies can effectively block the bacterial attachment to collagen. This data evidences that the extracellular, mature GehD promotes S. epidermidis attachment to collagenous surfaces and confirms that compositions containing GehD or antibodies thereto will be useful in methods of treating or preventing staphylococcal infections.

The ability of mature GehD to bind to collagen was revealed as we screened a S. epidermidis genomic library searching for collagen-binding MSCRAMMs. In contrast to S. aureus, adherence of S. epidermidis to extracellular matrix proteins has not been well characterized. It is known that S. epidermidis can adhere to fibrinogen, fibronectin, laminin (8) and vitronectin (7). The adherence to fibrinogen is mediated by protein adhesins such as Fbe (15) or SdrG (1), and attachment to vitronectin seems to be promoted by the autolysin AtlE. However, the proteins responsible for the interactions with collagen and fibronectin have previously remained undiscovered. Thus, to search for additional adhesins, we constructed a genomic expression library from the clinical isolate S. epidermidis 9491. To screen our library, we took advantage of a panel of 22 mAbs that were raised against Cna(151-318), the collagen-binding MSRAMM from S. aureus. Two of these monoclonals (11H11 and 1F6) cross-reacted to epitopes present on the surface of S. epidermidis cells. Therefore, we used mAbs 11H11, 1F6 and labeled collagen, to screen the expression library and isolate a collagen-binding clone. Surprisingly, the clone that bound to both mAbs and collagen expressed the mature version of GehD. This S. epidermidis extracellular lipase has the same overall organization as the other staphylococcal lipases GehC, Geh, Sal2 and Lip (3). These lipases appear to be synthesized as pre-proenzymes consisting of three major domains: signal peptide, propeptide and mature lipase. The signal peptide is essential for secretion and it is removed during export of the protein. The propeptide domain has been found to be important for efficient translocation and proteolytic stability during secretion (11). Previous data (12) suggests that GehD is similarly translated as a preproenzyme, and post-translationally processed into mature lipase. The size of this active, extracellular lipase is approx. 45 kDa.

Mature GehD was identified as a collagen-binding adhesin using mAbs raised against Cna(151-318). The amino acid sequences from GehD and Cna, however, do not display significant similarities. The predicted secondary structure of these two proteins also seems to be dissimilar: GehD is predicted to fold into a structure of at least 26% α-helix, whereas the CNA crystal structure shows it to be composed mostly of β-sheets (20). Despite the low amino acid sequence similarity and the finding that GehD and Cna may have very different predicted secondary structures, mAbs 1F6 and 11H11 recognize both proteins. This raises the possibility that the conformationally-dependent epitope present on Cna(151-318) recognized by 11H11 and 1F6 may also be found in GehD.

The mature form of GehD can be found associated to whole cells and in lysostaphin extracts from the cell wall. However, the typical motifs associated with the cell-wall anchored proteins found in most Gram-positive bacterial surface proteins are not present in the carboxy-terminus of the GehD protein. The nature of the association between GehD and the S. epidermidis cell surface is, at this moment, not understood.

Mutants of S. epidermidis 9 defective in GehD or GehC were used to examine the role of GehD in bacterial interactions with collagen. GehD can mediate bacterial attachment to immobilized collagen. This interaction was blocked by recombinant, mature GehD. In addition, antibodies raised against the mature GehD lipase inhibit the attachment of S. epidermidis9 to collagen. Both the gehc and gehD mutants show a decreased attachment to collagen, which raises the possibility that GehC might also interact with collagen. It is interesting to note that we did not find GehC in our library search for collagen adhesins. There are at least two possibilities that could explain this phenomenon: GehC might have a lower binding affinity for collagen, rendering a GehC-expressing clone very hard to detect. Alternatively, when generating the library, the GehC coding sequence could have been inserted in a different translation frame to that of the vector, thus, impeding its correct expression. The ability of recombinant GehC to bind to collagen has not been explored, but it is of future interest.

Amino acid sequence analysis has shown that GehC and GehD are 51% identical to each other. GehC is closely related to lipase Sal-2 from S. aureus NCTC 8530 (84% identity), whereas GehD has greater homologies to the S. aureus PS54 lipase, Geh (58% identity), and the lipase of S. haemolyticus, Lip (70%) identity (12). Although the staphylococcal lipases are a diverse group of enzymes, the predicted secondary structures contain conserved elements. It is also thought that these staphylococcal lipases might also have adhesive properties in addition to their lipolytic activities. The ability of this enzyme to be bi-functional may be indicative of its importance to S. epidermidis successful colonization and growth on both skin and artificial surfaces, and evidences that the compositions and antibodies to GehD in accordance with the present invention will be highly useful in methods of blocking bacterial attachment to collagen and in treating and preventing staphylococcal inventions and outbreaks.

### REFERENCES

1.Davis, S. L., S. Gurusiddappa, K. W. McCrea, S. Perkins, and M. H\ddot{oo}k. 2001. SdrG, a fibrinogen-binding bacterial adhesin of the microbial surface components recognizing adhesive matrix molecules subfamily from Staphylococcus epidermidis, targets the thrombin cleavage site in the Bβ chain. J. Biol. Chem. 276(30):27799-27805.

2.Garrett, D. O., E. Jochimsen, K. Murfitt, B. Hill, S. McAllister, P. Nelson, R. V. Spera, R. K. Sall, F. C. Tenover, J. Johnston, B. Zimmer, and W. R. Jarvis. 1999. The emergence of decreased susceptibility to vancomycin in Staphylococcus epidermidis [see comments]. Infect Control Hosp Epidemiol. 20(3):167-70.

3.Götz, F., H. M. Verheij, and R. Rosenstein. 1998. Staphylococcal lipases: molecular characterization, secretion, and processing. Chem. Phys. Lipids. 93(1-2):15-25.

4.Gribbon, E. M., W. J. Cunliffe, and K. T. Holland. 1993. Interaction of Propionibacterium acnes with skin lipids in vitro. J. Gen. Microbiol. 139:1745-1751.

5.Hedström, S. A. 1975. Lipolytic activity of Staphylococcus aureus strains from cases of human chronic osteomyelitis and other infections. Acta Pathol. Microbiol. Scan. Sect. B. Microbiol. 83:285-292.

6.Hedström, S. A., and P. Nilsson-Ehle. 1983. Triacylglycerol lipolysis by Staphylococcus aureus strains from furunculosis, pyomyosititis, impetigo and osteomyelitis. Acta Pathol. Microbiol. Scan. Sect. B Microbiol. 91:169-173.

7.Heilmann, C., M. Hussain, G. Peters, and F. Gotz. 1997. Evidence for autolysin-mediated primary attachment of Staphylococcus epidermidis to a polystyrene surface. Mol Microbiol. 24(5):1013-24.

8.Herrmann, M., P. Vaudaux, R. A. D. Pittet, P. D. Lew, F. Schumacher-Perdreau, G. Peters, and F. A. Waldvogel. 1988. Fibronectin, fibrinogen and laminin act as mediators of adherence of clinical staphylococcal isolates to foreign material. J. Infect. Dis. 158:693-701.

9.Hogt, A. H., J. Dankert, and J. Feijen. 1986. Adhesion of coagulase-negative staphylococci to methacrylate polymers and copolymers. J Biomed Mater Res. 20(4):533-45.

10.Laemmli, U. K. 1970. Cleavage of structural proteins during the assembly of the head of bacteriophage T4. Nature. 227:680-685.

11 .Liebl, W., and F. Götz. 1986. Studies on lipase directed export of Escherichia coli beta-lactamase in Staphylococcus carnosus. Mol. Gen. Genet. 204:166-173.

12.Longshaw, C. M., A. M. Farrell, J. D. Wright, and K. T. Holland. 2000. Identification of a second lipase gene, gehD, in Staphylococcus epidermidis: comparison of sequence with those of other staphylococcal lipases. Microbiology. 146:1419-1427.

13.Lowe, A. M., D. T. Beattie, and R. L. Deresiewicz. 1998. Identification of novel staphylococcal virulence genes by in vivo expression technology. Mol. Microbiol. 27:967-976.

14.Muller, E., J. Hubner, N. Gutierrez, S. Takeda, D. A. Goldmann, and G. B. Pier. 1993. Isolation and characterization of transposon mutants of Staphylococcus epidermidis deficient in capsular polysaccharide/adhesin and slime. Infect Immun. 61(2):551-8.

15.Nilsson, M., L. Frykberg, J. I. Flock, M. Lindberg, and B. Guss. 1998. A fibrinogen-binding protein of Staphylococcus epidermidis. Infect. Immun. 66:2666-2673.

16.Pace, C. N., F. Vajdos, L. Fee, G. Grimsley, and T. Gray. 1995. How to measure and predict the molar absorption coefficient of a protein. Protein Sci. 4(11):2411-2423.

17.Pascual, A., A. Fleer, N. A. Westerdaal, and J. Verhoef. 1986. Modulation of adherence of coagulase-negative staphylococci to Teflon catheters in vitro. Eur J Clin Microbiol. 5(5):518-22.

18.Rollof, J., S. A. Hedström, and P. Nilsson-Ehle. 1987. Lipolytic activity of Staphylococcus aureus strains from disseminated and localized infections. Acta Pathol. Mocrobiol. Immunol. Scand. Sect. B Microbiol. 95:109-113.

19.Symerski, J., J. M. Patti, M. Carson, K. House-Pompeo, M. Teale, D. Moore, L. Jin, L. J. DeLucas, M. H\ddot{oo}k, and S. V. L. Narayana. 1997. Structure of the collagen-binding domain from a Staphylococcus aureus adhesin. Nat. Struct. Biol. 10:833-838.

20.Symersky, J., J. M. Patti, M. Carson, K. House-Pompeo, M. Teale, D. Moore, L. Jin, A. Schneider, L. J. DeLucas, M. H\ddot{oo}k, and V. L. N. Sthanam. 1997. Structure of the collagen-binding domain from a Staphylococcus aureus adhesin. Nat. Struct. Biol. 4:833-838.

21.Tojo, M., N. Yamashita, D. A. Goldmann, and G. B. Pier. 1988. Isolation and characterization of a capsular polysaccharide adhesin from Staphylococcus epidermidis [published erratum appears in J Infect Dis 1988 July;158(1):268]. J Infect Dis. 167(4):713-22.

22.Visai, L., Y. Xu, F. Casolini, S. Rindi, M. H\ddot{oo}k, and P. Speziale. 2000. Monoclonal antibodies to CNA, a collagen-binding microbial surface component recognizing adhesive matrix molecules, detach Staphylococcus aureus from a collagen substrate. J. Biol. Chem. 275(51):39837-39845.

23.Yu, J., M. N. Montelius, M. Paulsson, I. Gouda, O. Larm, L. Montelius, and A. Ljungh. 1994. Adhesion of a coagulase-negative staphylococci and adsorption of plasma proteins to heparinized polymer surfaces. Biomaterials. 15:805-814.

What is claimed is:

1. A therapeutic composition for treating or preventing a staphylococcal infection comprising a collagen-binding GehD lipase in an amount effective to treat or prevent a staphylococcal infection in a human or animal patient in need of such treatment and a suitable vehicle, excipient or carrier.

2. The therapeutic composition of claim 1 wherein the GehD lipase is from Staphylococcus epidermidis.

3. The therapeutic composition of claim 1 wherein the GehD lipase is in the form of MGehD.

4. An antibody that can recognize collagen-binding GehD lipase from Staphylococcus epidermidis.

5. A therapeutic composition for treating or preventing a staphylococcal infection comprising an antibody according to claim 4 in an amount effective to treat or prevent a staphylococcal infection in a human or animal patient in need of such treatment and a suitable vehicle, excipient or carrier.

6. The antibody according to claim 4 wherein the antibody can recognize MGehD.

7. The antibody according to claim 4 wherein the antibody can recognize the amino acid sequence of SEQ ID NO:1.

8. A vaccine comprising an immunogenic amount of collagen-binding GehD lipase and a pharmaceutically acceptable vehicle, excipient or carrier.

9. The vaccine of claim 8 wherein the GehD lipase is from Staphylococcus epidermidis.

10. The vaccine of claim 8 wherein the GehD lipase is in the form of MGehD.

11. Antibody or antisera raised against GehD lipase.

12. A diagnostic kit for determining the presence of GehD proteins in a sample suspected of containing such proteins comprising an antibody according to claim 4, means to introduce the antibody to the sample, and a means for determining the presence of binding of the antibodies and GehD proteins in the sample.

13. A diagnostic kit for determining the presence of GehD antibodies in a sample suspected of containing said antibodies comprising isolated GehD proteins, means to introduce the proteins to the sample, and a means for determining the presence of binding of the GehD proteins and the antibodies to GehD in the sample.

14. A method of treating or preventing a staphylococcal infection in a human or animal patient in need of such treatment comprising administering to the patient isolated GehD lipase from S. epidermidis in an amount effective to treat or prevent a staphylococcal infection.

15. A method of treating or preventing a staphylococcal infection in a human or animal patient in need of such treatment comprising administering to the patient an antibody according to claim 4 in an amount effective to treat or prevent a staphylococcal infection.

16. A method of preventing binding of a staphylococcal bacteria to collagen in a human or animal patient comprising administering to the patient an antibody according to claim 4 in an amount sufficient to inhibit binding of staphylococci to collagen.

17. A method of reducing staphylococci infection of an indwelling medical device or implant comprising coating the medical device or implant with a GehD lipase in an amount effective to reduce or inhibit binding of staphylococci to the medical device or implant.

18. The method of claim 17 wherein the medical device is selected from the group consisting of vascular grafts, vascular stents, intravenous catheters, artificial heart valves, and cardiac assist devices.

19. A method of inducing an immunological response to GehD comprising administering to a patient a composition comprising an immunogenic amount of an isolated GehD lipase or active fragments thereof.

20. The method of claim 19 wherein the GehD lipase is from S. epidermidis.

21. The method of claim 19 wherein the GehD lipase is MGehD.


SEQUENCE LISTING (1 sequence)

SEQ ID NO: 1 - protein, 518 aa, Staphylococcus epidermidis
MAISRQLVDPNSQDSSDKKQHPSDQTQDSSSKGTQPKQSQSIEDRDKTVKQPSSKVHKIGNTKTDKTVKTNQKKQTSLTSPRVVKSKQTKHINQLTAQAQYKNQYPVVFVHGFVGLVGEDAFSMYPNYWGGTKYNVKQELTKLGYRVHEANVGAFSSNYDRAVELYYYIKGGRVDYGAAHAAKYGHKRYGRTYEGIMPDWEPGKKIHLVGHSMGGQTIRLMEHFLRNGNQEEIDYQRQYGGTVSDLFKGGQDNMVSTITTLGTPHNGTPAADKLGSTKFIKDTINRIGKIGGTKALDLELGFSQWGFKQQPNESYAEYAKRIANSKVWETEDQAVNDLTTAGAEKLNQMTTLNPNIVYTSYTGAATHTGPLGNEVPNIRQFPLFDLTSRVIGGDDNKNVRVNDGIVPVSSSLHPSDEAFKKVGMMNLATDKGIWQVRPVQYDWDHLDLVGLDTTDYKRTGEELGQFYMSMINNMLKVRRVKMVLHVSSDAAAQLYTRASQPELAPEDPEDLEHHHHHH